(19) United States
(12) Patent Application Publication
(10) Pub. No.: US 2005/0273868 A1
(43) Pub. Date: Dec. 8, 2005

(54) Methods and compositions for enhancing RISC activity in vitro and in vivo

(75) Inventor: Tariq M. Rana, Shrewsbury, MA (US)

Correspondence Address: LAHIVE & COCKFIELD, LLP., 28 STATE STREET, BOSTON, MA, 02109, US

(73) Assignee: UNIVERSITY OF MASSACHUSETTS, Boston, MA (US)

(21) Appl. No.: 11/060,851

(22) Filed: Feb. 17, 2005

Related U.S. Application Data
(60) Provisional application No. 60/545,558, filed on Feb. 17, 2004.

(51) Int. Cl. A01K 67/00; C12Q 1/68; C12N 15/85; C12N 5/08

(52) U.S. Cl. 800/8; 435/367; 435/6; 435/455

(57) ABSTRACT

The present invention provides methods of enhancing the efficacy and specificity of RNAi by priming RISC activity in cells, cell extracts, and organisms using priming agents such as siRNAs as well as other nucleic acids. The invention also provides priming agents, extracts and cells with high levels of primed RISC activity and therefore responsiveness to RNAi, and methods of using the same in research, diagnostic, and therapeutic applications.

### RELATED INFORMATION

The application claims priority to U.S. provisional patent application No. 60/545,558, filed on Feb. 17, 2004, the entire contents of which are hereby incorporated by reference.

### STATEMENT AS TO FEDERALLY SPONSORED RESEARCH

Funding for the work described herein was at least in part provided by the federal government under grant numbers AI 41404 and AI 43198, awarded by the United States National Institutes of Health and the National Institute of Allergy and Infectious Diseases.

The contents of any patents, patent applications, and references cited throughout this specification are hereby incorporated by reference in their entireties.

### BACKGROUND OF THE INVENTION

Double stranded RNA (dsRNA) induces a sequence-specific degradation of homologous mRNA in the cellular process known as RNA interference (RNAi). DsRNA-induced gene silencing has been observed in evolutionarily diverse organisms such as nematodes, flies, plants, fungi, and mammalian cells. Although the entire mechanism of RNAi has not yet been elucidated, several key elements have been identified. RNAi is initiated by an ATP-dependent processive cleavage of dsRNA into 21-23 nucleotide short interfering RNAs (siRNAs) by the DICER endonuclease. The siRNAs are then incorporated into an RNA-induced silencing complex (RISC). This protein and RNA complex is activated by ATP-dependent unwinding of the siRNA duplex. The activated RISC utilizes the antisense strand, also referred to as the guide strand, of the siRNA to recognize and cleave the corresponding mRNA, resulting in decreased expression of the protein encoded by the mRNA.

There recently has been a great deal of interest in the use of RNAi for basic research purposes and for the development of therapeutics to treat, e.g., disorders and/or diseases associated with unwanted or aberrant gene expression, however, siRNA effectiveness at mediating RNAi varies greatly, and can be affected by a number of factors including, but not limited to, the size of the siRNA, the size and nature of any overhangs, and the specificity of the siRNA. Even siRNAs having optimal length, overhangs and specificity, can be ineffective at mediating RNAi.

There is a need for further study of such systems. Moreover, there exists a need for the development of methods and reagents suitable for use in vitro and in vivo, in particular for use in developing human therapeutics.

### SUMMARY OF THE INVENTION

The present invention is based on the surprising discovery that cells previously thought to have low RISC activity, and therefore less responsiveness to RNAi or gene silencing, can actually be primed to have high RISC activity by first treating the cells (or organism) with a priming agent. The priming agents include chemically synthesized duplexed (annealed) nucleic acids, mixed nucleic acids (non-annealed), and single-stranded nucleic acids, including small 10 to 21 nucleotide siRNAs, non-canonical siRNAs, and even non-sequence specific nucleic acids. Cells primed (and resultant extracts) have high levels of RISC activity and therefore are now highly responsive to efficient and specific RNAi or gene silencing applications. Thus, the primed cells (or extracts thereof) have important in vitro use in performing, for example, high throughput RNAi/gene silencing screens for identifying the consequences of a specific gene activities which have been altered, for example, knocked-down.

Moreover, the invention has important in vivo applications in that cells exposed to or expressing a priming agent and organisms either derived from such cells or exposed to a priming agent, can be primed to be more responsive to RNAi/gene silencing. This discovery provides for first sensitizing cells, tissues, and whole organisms to then respond to more efficiently to RNAi/gene silencing therapies. This allows for research, diagnostic, and therapeutic approaches for determining/treating the consequences of in vivo gene activities using RNAi/gene silencing.

Accordingly, the invention has several advantages which include, but are not limited to, the following:
providing priming agents for increasing the level of RISC activity and RNAi responsiveness in cells, cell extracts, and whole organisms,
providing methods for increasing the level of RISC activity and RNAi responsiveness in cells, cell extracts, and whole organisms using such priming agents,
providing cells, cell extracts, and whole organisms having high RISC activity and RNAi responsiveness and in vitro screens using the same, and
providing in vivo methods for increasing the level of RISC activity and RNAi responsiveness in cells, tissues, and whole organisms for treating undesired gene activities.

Other features and advantages of the invention will be apparent from the following detailed description and claims.

### BRIEF DESCRIPTION OF THE DRAWINGS

FIG. 1 shows a schematic of the protocol for enhancing or “priming” the RISC activity of a cell, e.g., a mammalian cell, cell extract, or organism where the extract, cell, or organism is first exposed to a priming agent, e.g., a nucleic acid, and then subsequently to an RNAi agent, e.g., an siRNA, such that an increase in RNAi responsiveness (as a function of target destruction) is achieved. Graph depicts typical results.

FIG. 2 shows a panel of siRNA agents comprising non-canonical overhangs as a function of sense strand shortening and/or deletion of the dTdT end. The corresponding panel of antisense strand shortening and/or deletion of the dTdT end (not shown) mirror the panel of molecules shown except that the top strand (sense strand) is wild type and the alterations made to the sense strand are made to the lower antisense strand.

### DETAILED DESCRIPTION OF THE INVENTION

In order to provide a clear understanding of the specification and claims, the following definitions are conveniently provided below.

Definitions

So that the invention may be more readily understood, certain terms are first defined.

The term “RNA interference” (“RNAi”) or “RNAi activity” refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of a target gene(s).

The phrase “an siRNA having a sequence sufficiently complementary to a target mRNA sequence to direct target-specific RNA interference (RNAi)” refers to a siRNA having sequence sufficient to trigger the destruction of the target mRNA by the RNAi machinery or process.

The term “small interfering RNA” (“siRNA”) (also referred to in the art as “short interfering RNAs”) refers to an RNA (or RNA analog) including strand(s) (e.g., sense and/or antisense strands) comprising between about 10-50 nucleotides (or nucleotide analogs) which is capable of directing or mediating RNA interference.

The term “siRNA duplex” refers to an siRNA having complimentary stands, e.g., a sense strand and antisense strand, wherein the strands are base-paired or annealed (e.g., held together by hydrogen bonds).

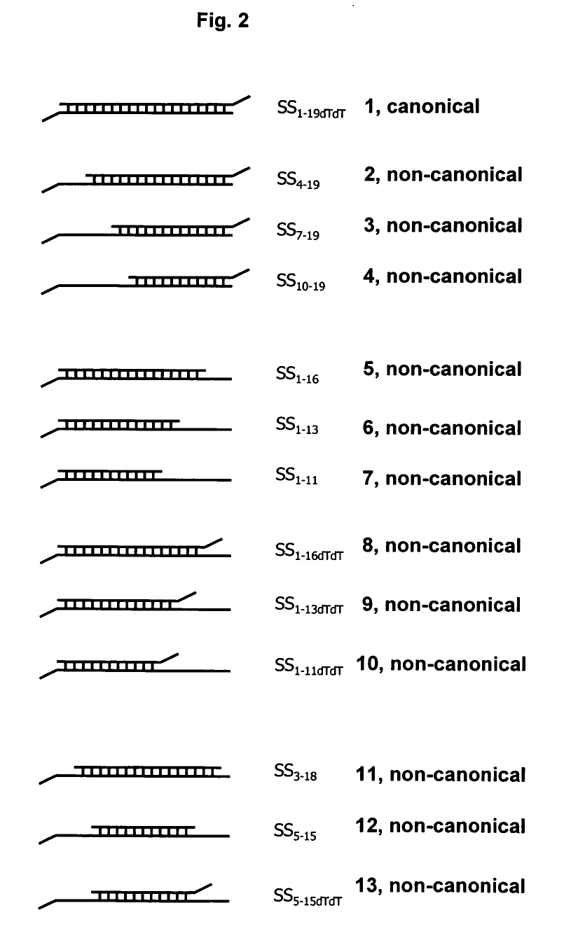

The term “non-canonical siRNA” refers to an siRNA having a non-canonical strand length(s) and/or overhang(s) (or end). A non-canonical strand length is typically less than 21 nucleotides but at least about 10 nucleotides. The term “non-canonical overhang” refers to the atypical overhang formed when the mixed, duplexed, or single stranded nucleic acids of the invention are aligned or annealed (in vitro or in vivo). The overhang(s) is distinguished from a “canonical” (or wild type) overhang of an siRNA in that the overhang lacks a 2-nucleotide overhang (e.g., dTdT) and/or one or more nucleotides. Accordingly, non-canonical overhangs include a 5′ overhang with 1, 2, 3, 4, 5, 6, 7, 8, 9, 10 or more nucleotide deletions (or truncations) and/or no dTdT (also referred to as a 5′ non-canonical overhang) as well as a 3′ overhang with 1, 2, 3, 4, 5, 6, 7, 8, 9, 10 or more nucleotide deletions and/or no dTdT (also referred to as a 3′ non-canonical overhang). Exemplary non-canonical siRNAs are shown in FIG. 2.

The term “target gene sequence” refers to a gene sequence encoding a nucleic acid or polypeptide gene product which can be targeted for degradation, e.g., by RNA interference or a RISC-mediated pathway. The target sequenced may be an artificial, recombinant, or naturally occurring sequence. In one embodiment, the sequence encodes a gene product that, when expressed, e.g., at aberrant levels, results in a undesired phenotype, disorder, or disease, in for example, a model organism or human subject.

The phrase “separately and temporally” refers to priming agents, and siRNAs of the invention that exist or are expressed as separate strands, e.g., a sense single-strand and an antisense single strand that are introduced, e.g., to an extract, cell, or organism as a non-annealed mixture or separately, i.e., unmixed, with, preferably, one strand being introduced first followed after a time interval (e.g., several minutes to about 1 hour or more, e.g., 24, 48, or 72 hours), the second strand.

The term “priming agent” refers to a compound, typically a nucleic acid, e.g., a oligonucleotide or single-stranded nucleic acid, mixture or annealed nucleic acid, siRNA, shRNA, non-canonical siRNAs, or even non-sequence specific nucleic acids, which can be used to enhance or “prime”, “program”, “activate”, or “trigger” an RNAi pathway, e.g., RISC activity, in a cell extract, cell, or organism. Typically, the priming agent is introduced or expressed in the cell using art recognized techniques.

The term “RISC” or “RNA induced silencing complex” refers to the nucleic acid and polypeptide components, e.g., Dicer, R2D2, and the Argonaute family of polypeptides, that interact to recognize target gene sequences, e.g., RNA molecules for targeted destruction or silencing. This activity is also referred to as “RISC activity” or “RNA induced silencing complex activity”.

The term “high level of activated RISC” refers to a level of RISC activity, e.g., as measured by target gene degradation, which is sufficiently elevated or above what is usual for a comparable/control extract, cell, or organism. For example, in mammalian cells, e.g., HeLa cells, the high level of RISC activity is calculated to be about 0.2 to about 1.9 nM or more for a single cell. Typically, the high level of activated RISC is achieved by priming a cell, cell extract, or organism by exposing the cell, cell extract, or organism to a priming agent as described herein. Changes in primed RISC activity as compared to a control result in a fold increase of 1.5, 2, 3, 4, 5, 10, 15, 20, or more.

The term “nucleic acid” and “single-stranded nucleic acid” refers to RNA or RNA molecules as well as DNA molecules. The term RNA refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively), or multi-stranded (e.g., double stranded, i.e., dsRNA and dsDNA, respectively), i.e., duplexed or annealed.

The term “modified nucleotide” or “modified nucleic acid(s)” refers to a non-standard nucleotide or nucleic acid, including non-naturally occurring ribonucleotides or deoxyribonucleotides. Preferred nucleotide analogs or nucleic acids are modified at any position so as to alter certain chemical properties, e.g., increase stability of the nucleotide or nucleic acid yet retain its ability to perform its intended function, e.g., have priming and/or RNAi activity. Examples include methylation at one or more bases, e.g., O-methylation, preferably 2′ O methylation (2′-O-Me), dyes which can be linked to the nucleic acid to provide for visual detection of the nucleic acid, and biotin moieties which can be used to purify the nucleic acid to which it is attached as well as any associated components bound to the biotinylated nucleic acid. Other examples of modified nucleotides/nucleic acids are described in Herdewijn, Antisense Nucleic Acid Drug Dev., 2000 Aug. 10(4):297-310; U.S. Pat. Nos. 5,858,988; 6,291,438; Eckstein, Antisense Nucleic Acid Drug Dev. 2000 Apr. 10(2): 117-21; Rusckowski et al. Antisense Nucleic Acid Drug Dev. 2000 Oct. 10(5):333-45; Stein, Antisense Nucleic Acid Drug Dev. 2001 Oct. 11(5): 317-25; Vorobjev et al. Antisense Nucleic Acid Drug Dev. 2001 Apr. 11(2):77-85; and U.S. Pat. No. 5,684,143.

A gene “involved” in a disorder includes a gene, the normal or aberrant expression or function of which effects or causes a disease or disorder or at least one symptom of the disease or disorder

The phrase “examining the function of a gene in a cell or organism” refers to examining or studying the expression, activity, function or phenotype arising therefrom. Various methodologies of the invention include a step that involves comparing a value, level, feature, characteristic, property, etc. to a “suitable control”, referred to interchangeably herein as an “appropriate control”.

A “suitable control” or “appropriate control” refers to any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a RISC level of activity or amount, target gene level or target gene degradation level, a transcription rate, mRNA level, translation rate, protein level, biological activity, cellular characteristic or property, genotype, phenotype, etc. can be determined prior to introducing a nucleic acid of the invention into a cell, cell extract, or organism.

The term “cell” refers to any eukaryotic cell which exhibits RNAi activity and includes, e.g., animal cells (e.g., mammalian cells, e.g., human or murine cells), plant cells, and yeast. The term includes cell lines, e.g., mammalian cell lines such as HeLa cells as well as embryonic cells, e.g., embryonic stem cells and collections of cells in the form of, e.g., a tissue.

The term “cell extract” refers to a lysate or acellular preparation of a cell as defined above and can be a crude extract or partially purified as well as comprise additional agents such as recombinant polypeptides, nucleic acids, and/or buffers or stabilizers.

The term “organism” refers to multicellular organisms such as, e.g., C. elegans, Drosophila, mouse, and human.

The term “vector” refers to a nucleic acid molecule (either DNA or RNA) capable of conferring the expression of a gene product when introduced into a host cell or host cell extract. In one embodiment, the vector allows for temporal or conditional expression of one or more nucleic acids of the invention, e.g., a priming agent, single strand, siRNA, non-canonical siRNA, or shRNA. The vector may be episomal or chromosomally (e.g., transgenically) integrated into the host cell genome.

### DETAILED DESCRIPTION

Overview

The present invention features cell extracts which mediate RNA interference (RNAi) where the extract is primed such that it has a high level of activated RISC relative to a suitable control. Preferred extracts of the invention are from cells of mammalian origin, for example, human origin, for example, embryonic cells, such as embryonic stem cells, or a cell line such as HeLa cells.

The cell extracts of the invention are primed though the use of a priming agent, such as an annealed siRNA duplex, a non-annealed siRNA duplex, non-canonical siRNA, a single siRNA strand, or a shRNA that encodes an siRNA strand or siRNA duplex.

Typically, the extracts of the invention comprise levels of activated RISC or RNA interference (RNAi) activity that are 2-fold, 5-fold, 10-fold, or 20-fold or greater or greater than the activity found in a suitable control.

The invention also provides priming agents suitable for activating RISC in a cell and include an annealed siRNA duplex, a non-annealed siRNA duplex, a single siRNA strand, and a shRNA that encodes an siRNA strand or siRNA duplex, in for example, a pharmaceutically acceptable carrier, or liposome.

The priming agents of the invention may also be expressed in a cell and therefore encoded in a vector, preferably a vector capable of conditional expression and/or tissue specific expression. The tet operator and operon is a preferred conditional expression system.

The invention also provides cells having a priming agent, for example, as expressed from a vector, maintained episomally or chromosomally integrated (e.g. transgenically) into the genome of the cell. Accordingly, organisms, for example transgenic organisms, may be derived or comprise such a cell, and include non-human transgenic organisms such as a transgenic mouse.

The invention also provides kits for carrying out the invention, e.g., making or using primed cells or cell extracts by providing instructions to the same and/or components such as priming agent, primed cells or extracts, e.g., mammalian cell extracts (e.g., HeLa cell extracts), or cells or organism primed or suitable for priming to have high levels of RISC activity.

Typically priming agents of the kit include, e.g., an annealed siRNA duplex, a non-annealed siRNA duplex, a non-canonical siRNA, a single siRNA strand, and a shRNA that encodes an siRNA strand or siRNA duplex.

The invention also provides cells having activated RISC produced by a process comprising exposing the cell to a sufficient amount of priming agent to activate the RISC, such that a high level of activated RISC, relative to a suitable control, is achieved.

Suitable priming agents for carrying out the process include, e.g., an annealed siRNA duplex, a non-annealed siRNA duplex, a non-canonical siRNA, a single siRNA strand, and/or a shRNA that encodes an siRNA strand or siRNA duplex.

Cells produced by the process are also within the scope of the invention and include, for example, mammalian cells, e.g., human cells, embryonic stem cells, human cell lines such as HeLa cells, and extracts or organisms derived from such cells as are appropriate.

Still further, methods of making primed cells and cell extracts are encompassed by the invention and include exposing the cell to a sufficient amount of priming agent to activate the RISC, such that a high level of activated RISC, relative to a suitable control, is achieved. The cells are typically lysed to obtain a primed lysate or optionally, for purifying or partially purifying the activated RISC or components thereof.

In another embodiment, the invention provides methods of mediating RNAi, the method comprising contacting RISC, an extract, a cell, or an organism to a priming agent, and exposing the RISC, an extract, cell, or organism to an siRNA such that target specific RNAi is capable of being achieved. Preferred extracts generated form the method include extracts from cells of mammalian origin, for example, human origin, for example, embryonic cells, such as embryonic stem cells, or a cell line such as HeLa cells. Wherein the method employs an organism, the organism may be, e.g., C. elegans, Drosophila, mouse, or human. In the case of a human, a priming of the human, may be a first step which is then followed by an RNAi step in order to achieve a therapeutic reduction in an undesired gene.

Accordingly, the invention provides methods for treating a disease or disorder associated with the activity of a protein specified by a target mRNA in a subject by administering to the subject a priming agent sufficient to activate RISC in one or more cells and administering an siRNA in an amount sufficient for degradation of the target mRNA to occur, thereby treating the disease or disorder associated with the polypeptide encoded by the target mRNA.

Still further, the invention encompasses research applications whereby, e.g., information about the function of a gene in a extract, cell, or organism is derived by exposing a primed extract, cell, or organism as described herein to an siRNA, maintaining the lysate, cell, or organism under conditions such that target-specific RNAi can occur, determining a characteristic or property of the extract, cell, or organism, and comparing the characteristic or property to a suitable control, the comparison yielding information about the function of the gene. Wherein the method employs an organism, the organism may be, e.g., C. elegans, Drosophila, mouse, or human.

Further details for carrying out various aspects of the invention are provided in the following subsections below.

1. Priming Agents

The present invention features nucleic acids such as “small interfering RNA molecules” (“siRNA molecules” or “siRNA” but also single and double stranded shRNAs and non-canonical siRNAs) which can be used as priming agents for enhancing the RISC activity of a cell, e.g., a mammalian cell. Typically, a priming agent, e.g., an siRNA molecule of the invention is a duplex consisting of a sense strand and complementary antisense strand, the antisense strand having sufficient complementarity to a target mRNA to mediate RNAi. Preferably, one strand is administered first to prime the cell, cell extract, or organism, with the second strand being added subsequently to carryout and complete the RNAi/gene silencing.

The siRNA strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed, however, non-canonical overhangs may also be used. Preferably, the siRNA molecule has a length from about 10-50 or more nucleotides, i.e., each strand comprises 10-50 nucleotides (or nucleotide analogs). More preferably, the siRNA molecule has a length from about 15-45 nucleotides. Even more preferably, the siRNA molecule has a length from about 18-25 nucleotides. The siRNA molecules of the invention further have a sequence that is “sufficiently complementary” to a target mRNA sequence to direct target-specific RNA interference (RNAi), as defined herein, i.e., the siRNA has a sequence sufficient to trigger the destruction of the target mRNA by the RNAi machinery or process. Non-canonical strand lengths may also be used.

2. Producing Priming Agents

Nucleic acid priming agents may be produced enzymatically or by partial/total organic synthesis. In one embodiment, the nucleic acids of the invention are prepared chemically. Methods of synthesizing nucleic acid molecules are known in the art, in particular, the chemical synthesis methods as de scribed in Verma and Eckstein (1998) Annul Rev. Biochem. 67:99-134. In another embodiment, the nucleic acids are produced enzymatically, e.g., by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). In one embodiment, the siRNAs are synthesized either in vivo, in situ, or in vitro. Endogenous RNA polymerase of the cell may mediate transcription in vivo or in situ, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the siRNA. Inhibition may be targeted by specific transcription in an organ, tissue, or cell type; stimulation of an environmental condition (e.g., infection, stress, temperature, chemical inducers); and/or engineering transcription at a developmental stage or age or by conditional expression from a vector or transgene having an inducible promoter or operon. A transgenic organism that expresses a nucleic acid priming agent RNA from a recombinant construct may be produced by introducing the construct into a zygote, an embryonic stem cell, or another multipotent cell derived from the appropriate organism.

3. Modified Priming Agents

The invention also features priming agents, e.g., small interfering RNAs (siRNAs) that include a sense strand and an antisense strand, wherein the antisense strand has a sequence sufficiently complementary to a target mRNA sequence to direct target-specific RNA interference (RNAi) and wherein the sense strand and/or antisense strand is modified by the substitution of modified nucleotides, such that in vivo stability is enhanced as compared to a corresponding unmodified siRNA. For example, the priming agent may be methylated, e.g., 2′O-methylated at one of more bases. Certain modifications confer useful properties to siRNA. For example, increased stability compared to an unmodified siRNA or a label that can be used, e.g., to trace the siRNA, to purify an siRNA, or to purify the siRNA and cellular components with which it is associated. For example, such modifications may be used to stabilize the first (priming) strand for enhancing RISC activity/RNAi responsiveness in a cell (or cell extract or organism) and improve its intracellular half-life for subsequent receipt of the second strand wherein RNAi/gene silencing can now progress. Certain modifications can also increase the uptake of the siRNA by a cell. For example, functional groups such as biotin are useful for affinity purification of proteins and molecular complexes involved in the RNAi mechanism. The invention also includes methods of testing modified siRNAs for retention of the ability to act as an siRNA (e.g., in RNAi) and methods of using siRNA derivatives, e.g., in order to purify or identify RISC components (see, e.g., PCT/US03/36551; PCT/US03/24595; and PCT/JUS03/30480).

Modifications have the added feature of enhancing properties such as cellular uptake of the siRNAs and/or stability of the siRNAs. Preferred modifications are made at the 2′ carbon of the sugar moiety of nucleotides within the siRNA. Also preferred are certain backbone modifications, as described herein. Also preferred are chemical modifications that stabilize interactions between base pairs, as described herein. Combinations of substitution are also featured. Preferred modifications maintain the structural integrity of the antisense siRNA-target mRNA duplex.

The present invention features modified siRNAs. siRNA modifications are designed such that properties important for in vivo applications, in particular, human therapeutic applications, are improved without compromising the RNAi activity of the siRNA molecules e.g., modifications to increase resistance of the siRNA molecules to nucleases. Modified siRNA molecules of the invention comprise a sense strand and an antisense strand, wherein the sense strand or antisense strand is modified by the substitution of at least one nucleotide with a modified nucleotide, such that, for example, in vivo stability is enhanced as compared to a corresponding unmodified siRNA, or such that the target efficiency is enhanced compared to a corresponding unmodified siRNA. Such modifications are also useful to improve uptake of the siRNA by a cell. Preferred modified nucleotides do not effect the ability of the antisense strand to adopt A-form helix conformation when base-pairing with the target mRNA sequence, e.g., an A-form helix conformation comprising a normal major groove when base-pairing with the target mRNA sequence.

Modified siRNA molecules of the invention (i.e., duplex siRNA molecules) can be modified at the 5′ end, 3′ end, 5′ and 3′ end, and/or at internal residues, or any combination thereof. Internal siRNA modifications can be, for example, sugar modifications, nucleobase modifications, backbone modifications, and can contain mismatches, bulges, or crosslinks. Also preferred are 3′ end, 5′ end, or 3′ and 5′ and/or internal modifications, wherein the modifications are, for example, cross linkers, heterofunctional cross linkers, dendrimer, nano-particle, peptides, organic compounds (e.g., fluorescent dyes), and/or photocleavable compounds.

In one embodiment, the siRNA molecule of the invention comprises one or more (e.g., about 1, 2, 3, 4, 5, 6, 7, 8, 9, 10, or more) end modifications. Modification at the 5′ end is preferred in the sense strand, and comprises, for example, a 5′-propylamine group. Modifications to the 3′ OH terminus are in the sense strand, antisense strand, or in the sense and antisense strands. A 3′ end modification comprises, for example, 3′-puromycin, 3′-biotin and the like.

In another embodiment, the siRNA molecule of the invention comprises one or more (e.g., about 1, 2, 3, 4, 5, 6, 7, 8, 9, 10, or more) crosslinks, e.g., a crosslink wherein the sense strand is crosslinked to the antisense strand of the siRNA duplex. Crosslinkers useful in the invention are those commonly known in the art, e.g., psoralen, mitomycin C, cisplatin, chloroethylnitrosoureas and the like. A preferred crosslink of the invention is a psoralen crosslink. Preferably, the crosslink is present downstream of the cleavage site referencing the antisense strand, and more preferably, the crosslink is present at the 5′ end of the sense strand.

In another embodiment, the siRNA molecule of the invention comprises one or more (e.g., about 1, 2, 3, 4, 5, 6, 7, 8, 9, 10, or more) sugar-modified nucleotides. Sugar-modifed nucleotides useful in the invention include, but are not limited to: 2′-fluoro modified ribonucleotide, 2′-OMe modified ribonucleotide, 2′-deoxy ribonucleotide, 2′-amino modified ribonucleotide and 2′-thio modified ribonucleotide. The sugar-modified nucleotide can be, for example, 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine or 2′-amino-butyryl-pyrene-uridine. A preferred sugar-modified nucleotide is a 2′-deoxy ribonucleotide. Preferably, the 2′-deoxy ribonucleotide is present within the sense strand and, for example, can be upstream of the cleavage site referencing the antisense strand or downstream of the cleavage site referencing the antisense strand. A preferred sugar-modified nucleotide is a 2′-fluoro modified ribonucleotide. Preferably, the 2′-fluoro ribonucleotides are in the sense and antisense strands. More preferably, the 2′-fluoro ribonucleotides are every uridine and cytidine.

In another embodiment, the siRNA molecule of the invention comprises one or more (e.g., about 1, 2, 3, 4, 5, 6, 7, 8, 9, 10, or more) nucleobase-modified nucleotides. Nucleobase-modified nucleotides useful in the invention include, but are not limited to: 5-bromo-uridine, 5-iodo-uridine, 5-methyl-cytidine, ribo-thymidine, 2-aminopurine, 5-fluoro-cytidine, and 5-fluoro-uridine, 2,6-diaminopurine, 4-thio-uridine; and 5-amino-allyl-uridine and the like.

In another embodiment, the siRNA molecule of the invention comprises one or more (e.g., about 1, 2, 3, 4, 5, 6, 7, 8, 9, 10, or more) backbone-modified nucleotides, for example, a backbone-modified nucleotide containing a phosphorothioate group. The backbone-modified nucleotide is within the sense strand, antisense strand, or preferably within the sense and antisense strands.

In another embodiment, the siRNA molecule of the invention comprises a sequence wherein the antisense strand and target mRNA sequences comprise one or more (e.g., about 1, 2, 3, 4, 5, 6, 7, 8, 9, 10, or more) mismatches. Preferably, the mismatch is downstream of the cleavage site referencing the antisense strand. More preferably, the mismatch is present within 1-6 nucleotides from the 3′ end of the antisense strand. In another embodiment, the siRNA molecule of the invention comprises a bulge, e.g., one or more (e.g., about 1, 2, 3, 4, 5, 6, 7, 8, 9, 10, or more) unpaired bases in the duplex siRNA. Preferably, the bulge is in the sense strand.

In another embodiment, the siRNA molecule of the invention comprises any combination of two or more (e.g., about 2, 3, 4, 5, 6, 7, 8, 9, 10, or more) siRNA modifications as described herein. For example, a siRNA molecule can comprise a combination of two sugar-modified nucleotides, wherein the sugar-modified nucleotides are 2′-fluoro modified ribonucleotides, e.g., 2′-fluoro uridine or 2′-fluoro cytidine, and 2′-deoxy ribonucleotides, e.g., 2′-deoxy adenosine or 2′-deoxy guanosine. Preferably, the 2′-deoxy ribonucleotides are in the antisense strand, and, for example, can be upstream of the cleavage site referencing the antisense strand or downstream of the cleavage site referencing the antisense strand. Preferably, the 2′-fluoro ribonucleotides are in the sense and antisense strands. More preferably, the 2′-fluoro ribonucleotides are every uridine and cytidine.

The invention is also related to the discovery that certain characteristics of siRNA are necessary for activity and that modifications can be made to an siRNA to alter physicochemical characteristics such as stability in a cell and the ability of an siRNA to be taken up by a cell. Accordingly, the invention includes siRNA derivatives; siRNAs that have been chemically modified and retain activity in RNA interference (RNAi). The invention also includes a dual fluorescence reporter assay (DFRA) that is useful for testing the activity of siRNAs and siRNA derivatives.

Accordingly, the invention includes an siRNA derivative that includes an siRNA having two complementary strands of nucleic acid, such that the two strands are crosslinked, a 3′ OH terminus of one of the strands is modified, or the two strands are crosslinked and modified at the 3′OH terminus. The siRNA derivative can contain a single crosslink (e.g., a psoralen crosslink). In some embodiments, the siRNA derivative has a biotin at a 3′ terminus (e.g., a photocleavable biotin), a peptide (e.g., a Tat peptide), a nanoparticle, a peptidomimetic, organic compounds (e.g., a dye such as a fluorescent dye), or dendrimer.

4. Selecting a Gene Target

In one embodiment, the target gene sequence or mRNA of the invention encodes the amino acid sequence of a cellular protein, e.g., a protein involved in cell growth or suppression, e.g., a nuclear, cytoplasmic, transmembrane, membrane-associated protein, or cellular ligand. In another embodiment, the target mRNA of the invention specifies the amino acid sequence of an extracellular protein (e.g., an extracellular matrix protein or secreted protein). Typical classes of proteins are developmental proteins, cancer gene such as oncogenes, tumor suppressor genes, and enzymatic proteins, such as topoisomerases, kinases, and telomerases.

In a preferred aspect of the invention, the target mRNA molecule of the invention specifies the amino acid sequence of a protein associated with a pathological condition. By modulating the expression of the foregoing proteins, valuable information regarding the function of such proteins and therapeutic benefits which may be obtained from such modulation can be obtained.

5. Determining Gene Target Sequence Identity

The target RNA cleavage reaction guided by siRNAs (e.g., by siRNAs) is highly sequence specific. In general, siRNA containing a nucleotide sequences identical to a portion of the target gene are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Moreover, not all positions of a siRNA contribute equally to target recognition. Mismatches in the center of the siRNA are most critical and essentially abolish target RNA cleavage. Mismatches upstream of the center or upstream of the cleavage site referencing the antisense strand are tolerated but significantly reduce target RNA cleavage. Mismatches downstream of the center or cleavage site referencing the antisense strand, preferably located near the 3′ end of the antisense strand, e.g. 1, 2, 3, 4, 5 or 6 nucleotides from the 3′ end of the antisense strand, are tolerated and reduce target RNA cleavage only slightly.

Sequence identity may determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

Greater than 90% sequence identity, e.g., 91%, 92%, 93%, 94%, 95%, 96%, 97%, 98%, 99% or even 100% sequence identity, between the siRNA and the portion of the target gene is preferred. Alternatively, the siRNA may be defined functionally as a nucleotide sequence (or oligonucleotide sequence) that is capable of hybridizing with a portion of the target gene transcript. Examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

6. Efficacy Assays

The invention features methods of assaying the ability of a compound of the invention (e.g., a siRNA, candidate RNAi derivative, modified siRNA, etc.) to modulate (e.g., inhibit) expression of a target RNA using a dual fluorescence system. The assay may be used to determine the amount of improved RISC activity after priming the cell. Other assay systems known in the art that measure the efficacy of an siRNA can be modified as described herein to evaluate whether a modified siRNA is also a priming agent.

A compound of the invention (e.g., a priming agent, a siRNA, candidate priming agent, candidate RNAi derivative, modified siRNA, etc.) can be tested for its ability to improve a cell or cell extract RISC activity and responsiveness in inhibiting expression of a targeted gene. For example, candidate RNAi derivatives that can inhibit such expression are identified as siRNA derivatives. Any system in which RNAi activity can be detected can be used to test the activity of a compound of the invention (e.g., a siRNA, candidate priming agent, candidate RNAi derivative, modified siRNA, etc.). In general, a system in which RNAi activity can be detected is incubated in the presence and absence of a compound of the invention (e.g., a siRNA, candidate priming agent, candidate RNAi derivative, modified siRNA, etc.).

The invention includes a dual fluorescence reporter gene assay (DFRG assay) that can be used to test a compound of the invention (e.g., a priming agent, candidate priming agent, a siRNA, non-canonical siRNA, candidate RNAi derivative, modified siRNA, etc.). The DFRG assay can also be used, for example, to test the ability of these and other types of compounds to inhibit expression of a targeted gene. Technical details of the assay are provided in PCT/US03/30480 which is incorporated by reference in its entirety.

7. Methods of Introducing Priming Agents into Cells

Physical methods of introducing nucleic acids include injection of a solution containing the nucleic acid, bombardment by particles covered by the nucleic acid, soaking the cell or organism in a solution of the nucleic acid, or electroporation of cell membranes in the presence of the nucleic acid. A viral construct packaged into a viral particle would accomplish both efficient introduction of an expression construct into the cell and transcription of nucleic acid encoded by the expression construct. Other methods known in the art for introducing nucleic acids to cells may be used, such as lipid-mediated carrier transport, chemical-mediated transport, such as calcium phosphate, and the like. Thus the nucleic acid may be introduced along with components that perform one or more of the following activities: enhance nucleic acid uptake by the cell, inhibit annealing of single strands, stabilize the single strands, or other-wise increase inhibition of the target gene.

Nucleic acid may be directly introduced into the cell (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The cell with the target gene may be derived from or contained in any organism. The organism may a plant, animal, protozoan, bacterium, virus, or fungus. The plant may be a monocot, dicot or gymnosperm; the animal may be a vertebrate or invertebrate. Preferred microbes are those used in agriculture or by industry, and those that are pathogenic for plants or animals.

Alternatively, vectors, e.g., transgenes encoding a priming agent/siRNA of the invention can be engineered into a host cell or transgenic animal using art recognized techniques.

8. Primed Cells/Organisms/Lysates Therefrom and Uses Therefore

A further preferred use for the agents of the present invention (or vectors or transgenes encoding same) is a functional analysis to be carried out in eukaryotic cells, or eukaryotic non-human organisms, preferably mammalian cells or organisms, e.g., rodents, e.g. rats and mice, NIH3T3, Hep2, and preferably primate cells, e.g., COS cells, monkey kidney cells, and most preferably human cells, e.g. human primary cells, such as fibroblasts, endothelial cells, embryonic stem cells, bone marrow cells, erythroid, myeloid, or lymphoid cells, fetal cells, as well as human cell lines, such 293 cells and HeLa cells, many of which are publicly available through, e.g., the American Type Culture Collection (ATCC), P.O. Box 1549, Manassas, Va. 20108.

By administering a suitable priming agent/RNAi agent which is sufficiently complementary to a target mRNA sequence to direct target-specific RNA interference, a specific knockout or knockdown phenotype can be obtained in a target cell, e.g. in a cell lysate or extract, culture, or in a target organism.

Cell lysates or extracts can be made as described herein using a modified Dignam protocol. Methods of making cell lysates, e.g., nuclear and/or cytoplasmic cell lysates as well as organelle enriched lysates are well know in the art (see, e.g., Sambrook, Fritsch and Maniatis, Molecular Cloning: Cold Spring Harbor Laboratory Press (1989); and Current Protocols in Molecular Biology, eds. Ausubel et al., John Wiley & Sons (1992)).

Thus, a further subject matter of the invention is a eukaryotic cell or a eukaryotic non-human organism exhibiting a target gene-specific knockout or knockdown phenotype comprising a fully or at least partially deficient expression of at least one endogenous target gene wherein said cell or organism is transfected with at least one vector comprising DNA encoding an RNAi agent capable of inhibiting the expression of the target gene. It should be noted that the present invention allows a target-specific knockout or knockdown of several different endogenous genes due to the specificity of the RNAi agent.

Gene-specific knockout or knockdown phenotypes of cells or non-human organisms, particularly of human cells or non-human mammals may be used in analytic to procedures, e.g. in the functional and/or phenotypical analysis of complex physiological processes such as analysis of gene expression profiles and/or proteomes. Preferably the analysis is carried out by high throughput methods using oligonucleotide based chips.

9. Screening Assays

The methods of the invention are also suitable for use in methods to identify and/or characterize RNAi agents, pharmacological agents, e.g. identifying new RNAi agents, pharmacological agents from a collection of test substances and/or characterizing mechanisms of action and/or side effects of known RNAi agents or pharmacological agents.

Thus, the present invention also relates to a system, for example, a high throughput system (HTS), for identifying and/or characterizing pharmacological agents acting on at least one target protein comprising: a eukaryotic cell, cell extract, or a eukaryotic non-human organism primed or capable of being primed and expressing at least one endogenous target gene coding for a target protein, at least one priming/RNAi agent molecule capable of enhancing RISC activity or RNA responsiveness and inhibiting the expression of at least one endogenous target gene, and a test substance or a collection of test substances wherein the properties of the test substance or collection of test substances are to be identified and/or characterized.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

In a preferred embodiment, the library is a natural product library, e.g., a library produced by a bacterial, fungal, or yeast culture. In another preferred embodiment, the library is a synthetic compound library.

This invention is further illustrated by the following examples which should not be construed as limiting.

10. Transgenic Organisms

Engineered priming/RNAi agents of the invention can be expressed in transgenic animals. These animals represent a model system for the study of disorders that are caused by, or exacerbated by, overexpression or underexpression (as compared to wildtype or normal) of nucleic acids (and their encoded polypeptides) targeted for destruction by the RNAi agents, e.g., siRNAs and shRNAs, and for the development of therapeutic agents that modulate the expression or activity of nucleic acids or polypeptides targeted for destruction.

Transgenic animals can be farm animals (pigs, goats, sheep, cows, horses, rabbits, and the like), rodents (such as rats, guinea pigs, and mice), non-human primates (for example, baboons, monkeys, and chimpanzees), and domestic animals (for example, dogs and cats). Invertebrates such as Caenorhabditis elegans or Drosophila can be used as well as non-mammalian vertebrates such as fish (e.g., zebrafish) or birds (e.g., chickens).

Engineered RNA precursors with stems of 18 to 30 nucleotides in length are preferred for use in mammals, such as mice. A transgenic founder animal can be identified based upon the presence of a transgene that encodes the new RNA precursors in its genome, and/or expression of the transgene in tissues or cells of the animals, for example, using PCR or Northern analysis. Expression is confirmed by a decrease in the expression (RNA or protein) of the target sequence.

Methods for generating transgenic animals include introducing the transgene into the germ line of the animal. One method is by microinjection of a gene construct into the pronucleus of an early stage embryo (e.g., before the four-cell stage; Wagner et al., 1981, Proc. Natl. Acad. Sci. USA 78:5016; Brinster et al., 1985, Proc. Natl. Acad. Sci. USA 82:4438). Alternatively, the transgene can be introduced into the pronucleus by retroviral infection. A detailed procedure for producing such transgenic mice has been described (see e.g., Hogan et al., Manipulating the Mouse Embryo. Cold Spring Harbor Laboratory, Cold Spring Harbor, N.Y. (1986); U.S. Pat. No. 5,175,383 (1992)). This procedure has also been adapted for other animal species (e.g., Hammer et al., 1985, Nature 315:680; Murray et al., 1989, Reprod Fert. Devl. 1:147; Pursel et al., 1987, Vet. Immunol. Histopath. 17:303; Rexroad et al., 1990, J. Reprod. Fert. 41 (suppl): 119; Rexroad et al., 1989, Molec. Reprod. Devl. 1:164; Simons et al., 1988, BioTechnology 6:179; Vize et al., 1988, J. Cell. Sci. 90:295; and Wagner, 1989, J. Cell. Biochem. 13B (suppl): 164). Clones of the non-human transgenic animals described herein can be produced according to the methods described in Wilmut et al. ((1997) Nature, 385:810-813) and PCT publication Nos. WO 97/07668 and WO 97/07669.

11. Methods of Treatment

The present invention provides for both prophylactic and therapeutic methods of treating a subject at risk of (or susceptible to) a disorder or having a disorder associated with aberrant or unwanted target gene expression or activity. In one embodiment, the subject is primed with a priming agent, and then administered an siRNA for suppressing the expression of an the undesired gene product. It is understood that “treatment” or “treating” as used herein, is defined as the application or administration of a therapeutic agent (e.g., a RNAi agent or vector or transgene encoding same) to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

12. Prophylactic Methods

In another aspect, the invention provides a method for preventing in a subject, a disease or condition associated with an aberrant or unwanted target gene expression or activity, by administering to the subject a therapeutic agent (e.g., a RNAi agent or vector or transgene encoding same). If appropriate, subjects are first treated with a priming agent so as to be more responsive to the subsequent RNAi therapy. Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

13. Therapeutic Methods

In yet another aspect, the invention pertains to methods of modulating target gene expression, protein expression or activity for therapeutic purposes. Accordingly, in an exemplary embodiment, the modulatory method of the invention involves contacting a cell capable of expressing target gene with a therapeutic agent (e.g., a priming agent, RNAi agent or vector or transgene encoding same) that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent), in vivo (e.g., by administering the agent to a subject), or ex vivo. Typically, subjects are first treated with a priming agent so as to be more responsive to the subsequent RNAi therapy. As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

14. Pharmacogenomics

The therapeutic agents (e.g., a RNAi agent or vector or transgene encoding same) of the invention can be administered to individuals to treat (prophylactically or therapeutically) disorders associated with aberrant or unwanted target gene activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266

15. Pharmaceutical Compositions

The invention pertains to uses of the above-described agents for therapeutic treatments as described infra. Accordingly, the modulators of the present invention can be incorporated into pharmaceutical compositions suitable for administration. Such compositions typically comprise the nucleic acid molecule, e.g., priming agent, and together or separately, an RNAi agent, e.g., an siRNA agent for carrying out gene silencing, and, optionally, a protein, antibody, or modulatory compound, if appropriate, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration.

The use of such media and agents for pharmaceutically active substances is well known in the art. Except insofar as any conventional media or agent is incompatible with the active compound, use thereof in the compositions is contemplated. Supplementary active compounds can also be incorporated into the compositions.

### Exemplification

Throughout the examples, the following materials and methods were used unless otherwise stated.

Materials and Methods

In general, the practice of the present invention employs, unless otherwise indicated, conventional techniques of nucleic acid chemistry, recombinant DNA technology, molecular biology, biochemistry, and cell and cell extract preparation. See, e.g., DNA Cloning, Vols. 1 and 2, (D. N. Glover, Ed. 1985); Oligonucleotide Synthesis (M. J. Gait, Ed. 1984); Oxford Handbook of Nucleic Acid Structure, Neidle, Ed., Oxford Univ Press (1999); RNA Interference: The Nuts & Bolts of siRNA Technology, by D. Engelke, DNA Press, (2003); Gene Silencing by RNA Interference: Technology and Application, by M. Sohail, CRC Press (2004); Sambrook, Fritsch and Maniatis, Molecular Cloning: Cold Spring Harbor Laboratory Press (1989); and Current Protocols in Molecular Biology, eds. Ausubel et al., John Wiley & Sons (1992). See also PCT/US03/36551 (Attorney Docket No. UMY-041PC); PCT/IUS03/24595 (Attorney Docket No. UMY-061PC); and PCT/US03/30480 (Attorney Docket No. UMY-062PC), of which all are incorporated in their entireties by reference herein.

siRNA Preparation

21-nucleotide RNAs were chemically synthesized as 2′ bis(acetoxyethoxy)-methyl ether-protected oligos by Dharmacon (Lafayette, Colo.). Synthetic oligonucleotides were deprotected, annealed and purified as described by the manufacturer. Successful duplex formation was confirmed by 20% non-denaturing polyacrylamide gel electrophoresis (PAGE). All siRNAs were stored in DEPC (0.1% diethyl pyrocarbonate)-treated water at −80° C. The sequences of GFP or RFP target-specific siRNA duplexes were designed according to the manufacturer's recommendation and subjected to a BLAST search against the human genome sequence to ensure that no endogenous genes of the genome were targeted.

Culture and Transfection of Cells

HeLa cells were maintained at 37° C. in Dulbecco's modified Eagle's medium (DMEM, Invitrogen) supplemented with 10% fetal bovine serum (FBS), 100 units/ml penicillin and 100 μg/ml streptomycin (Invitrogen). Cells were regularly passaged at sub-confluence and plated 16 hr before transfection at 70% confluency. Lipofectamine (Invitrogen)-mediated transient cotransfections of reporter plasmids and siRNAs were performed in duplicate 6-well plates as described by the manufacturer for adherent cell lines. A transfection mixture containing 0.16-0.66 μg pEGFP-C1 and 0.33-1.33 μg pDsRed1-N1 reporter plasmids (Clontech), various amounts of siRNA (1.0 nM-200 nM), and 10 μl lipofectamine in 1 ml serum-reduced OPTI-MEM (Invitrogen) was added to each well. Cells were incubated in transfection mixture for 6 hours and further cultured in antibiotic-free DMEM. Cells were treated under same conditions without siRNA for mock experiments. At various time intervals, the transfected cells were washed twice with phosphate buffered saline (PBS, Invitrogen), flash frozen in liquid nitrogen, and stored at −80° C. for reporter gene assays.

In Vivo Fluorescence Analysis

pEGFP-C1, pDsRed1-N1 reporter plasmids and 50 nM siRNA were cotransfected into HeLa cells by lipofectamine as described above except that cells were cultured on 35 mm plates with glass bottoms (MatTek Corporation, Ashland Mass.) instead of standard 6-well plates. Fluorescence in living cells was visualized 48 hours post transfection by conventional fluorescence microscopy (Zeiss). For GFP and RFP fluorescence detection, FITC and CY3 filters were used, respectively.

Dual Fluorescence Efficacy Assay

Was carried out as described in PCT/IUS03/30480. Briefly, HeLa cells were maintained at 37° C. in Dulbecco's modified Eagle's medium (DMEM, Invitrogen) supplemented with 10% fetal bovine serum (FBS), 100 units/ml penicillin, and 100 μg/ml streptomycin (Invitrogen). Cells were regularly passaged at subconfluence and plated 16 hr before transfection at 70% confluency. Lipofectamine (Invitrogen)-mediated transient cotransfections of reporter plasmids and siRNAs were performed in duplicate 6-well plates. A transfection mixture containing 0.16 μg pEGFP-C1 and 0.33 μg pDsRed2-N1 reporter plasmids (Clontech), various amount of siRNA (From 0.5 nM to 400 nM), and 10 μl lipofectamine in 1 ml serum-reduced OPTI-MEM (Invitrogen) was added to each well. Cells were incubated in transfection mixture for 6 hr and further cultured in antibiotic-free DMEM. Cells were treated under the same conditions without siRNA for mock experiments. At various time intervals, the transfected cells were washed twice with phosphate-buffered saline (PBS, Invitrogen), flash frozen in liquid nitrogen, and stored at −80° C. for reporter gene assays.

Fluorescence of GFP in cell lysates was detected by exciting at 488 nm and recording from 498-650 nm. The spectrum peak at 507 nm represents the fluorescence intensity of GFP. Fluorescence of RFP2 in the same cell lysates was detected by exciting at 568 nm and recording from 588 nm-650 nm. The spectrum peak at 583 nm represents the fluorescence intensity of RFP2. The fluorescence intensity ratio of target (EGFP) to control (RFP2) fluorophore was determined in the presence of siRNA duplex and normalized to that observed in the mocked treated cells. Normalized ratios less than 1.0 indicates specific interference.

Preparation of Cell Extracts

HeLa cell cytoplasmic extract was prepared following the Dignam protocol for isolation of HeLa cell nuclei (Dignam et al., 1983). The cytoplasmic fraction was dialysed against cytoplasmic extract buffer (20 mM Hepes, pH 7.9, 100 mM KCl, 2001 μM EDTA, 500 μM DTT, 500 μM PMSF, 2 mM MgCl₂ 10% glycerol). The extract was stored frozen at −70° C. after quick-freezing in liquid nitrogen. The protein concentration of HeLa cytoplasmic extract varied between 4 to 5 mg/ml as determined by using a BioRad protein assay kit.

Preparation of Primed Mammalian Cells and Cell Extracts Having High RISC Activity

Cells were transfected with chemically synthesized single strand (sense or antisense) or duplex siRNAs (Dharmacon). After 24 h of transfection, cells were harvested to prepare cell extracts. Cytoplasm from HeLa cells was prepared following the Dignam protocol for isolation of HeLa cell nuclei (Dignam et al. 1983). The cytoplasmic fraction was dialyzed against cytoplasmic extract buffer (20 mM Hepes, pH 7.9, 100 mM KCl, 200 μM EDTA, 500 μM DTT, 500 μM PMSF, 2 mM MgCl₂, 10% glycerol). The extract was frozen at −70° C. after quick-freezing in liquid nitrogen. The protein concentration of HeLa cytoplasmic extract varied between 4 to 5 mg/ml as determined by Biorad protein assay kit.

Preparation of Cap-Labeled Target mRNA

For mapping of the target RNA cleavage, a 124 nt EGFP transcript, corresponding to nts 195-297 relative to the start codon followed by the 21 nt complement of the SP6 promoter sequence, was amplified from template pEGFP-C1 by PCR using 5′ primer CCTAATACGACTCACTATAGGACCTACGGCGTGCAGTGC (T7 promoter underlined) and 3′ primer TTGATTTAGGTGACACTATAGATGGTGCGCTCCTGGACGT (SP6 promoter underlined). His-tagged mammalian capping enzyme was expressed in E. coli from a recombinant plasmid and purified to homogeneity. Guanylyltransferase labeling was performed by incubating 1 nmole of transcripts with 50 pmole his-tagged mammalian capping enzyme in the 100 μl capping reaction containing 50 mM Tris-HCl (pH 8.0), 5 mM DTT, 2.5 mM MgCl₂, 1 U/μl RNasin RNase inhibitor (Promega) and [α-³²P]GTP at 37° C. for 1 h. Reactions were chased for 30 min by supplementing GTP concentration to 100 μM. Cap-labeled target mRNA were resolved on 10% polyacrylamide-7 M urea gel and purified.

In Vitro Target mRNA Cleavage Assay

siRNA-mediated cleavage of target mRNA in human cytoplasmic extract was performed as described (Martinez et al. 2002) with some modifications. siRNA duplexes were pre-incubated in HeLa cytoplasmic extract at 37° C. for 15 min prior to addition of the 124 nt cap-labeled target mRNA generated as described above. After addition of all components, final concentrations were 500 nM siRNA, 50 nM target mRNA, 1 mM ATP, 0.2 mM GTP, 1 U/μl RNasin, 30 μg/ml creatine kinase, 25 mM creatine phosphate, and 50% S100 extract. Incubation was continued for 1.5 h. Cleavage reactions were stopped by the addition of 8 volumes of proteinase K buffer (200 mM Tris-HCl [pH 7.5], 25 mM EDTA, 300 mM NaCl, and 2% w/v SDS). Proteinase K, dissolved in 50 mM Tris-HCl [pH 8.0], 5 mM CaCl₂, and 50% glycerol, was added to a final concentration of 0.6 mg/ml. Reaction products were extracted with phenol/chloroform/isoamyl alcohol (25:24:1), chloroform and precipitated with 3 volumes of ethanol. Samples were separated on 8% polyacrylamide-7 M Urea gels.

### EXAMPLE 1

### In Vitro Methods for Activating RISC in Mammalian Cells

The following example describes methods for activating RISC activity in mammalian cells by first treating the cells with a priming agent whereby the resultant cells and extracts that can be derived therefrom, are substantially improved for carrying out RNAi on a given target gene.

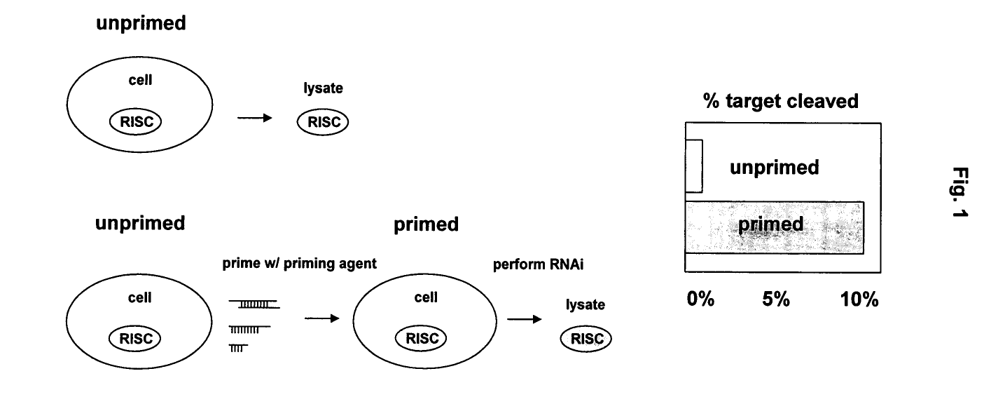

Briefly, cells were transfected with a nucleic acid priming agent as described above. Cells or cell extracts where then isolated/prepared alongside appropriate controls and challenged in an siRNA-mediated cleavage assay (as described above) to determine the level of RISC activity in the primed versus unprimed cells/extracts as a function of specific gene target degradation. Unprimed human HeLa cell extracts where determined to have only 0.1-1.0% gene target cleavage activity whereas primed HeLa cell extracts were determined to have 10% or more gene target cleavage activity (see FIG. 1).

Accordingly, these results indicate that priming agents can be used to activate RISC activity to a high level in mammalian cells, in particular, human cells, whereby the cells are now substantially responsive to RNAi/gene silencing techniques.

### EXAMPLE 2

### In Vivo Methods for Activating Risc in a Mammal

The following example describes methods for activating RISC activity in a whole organism by first exposing the organism to a priming agent whereby the organism is more responsive to RNAi/gene silencing techniques.

Briefly, a model organism is chosen and exposed to a priming agent. Preferably, the organism is a mouse which has been transgenically altered to express a priming agent, the priming agent being in the form of, e.g., an expressible nucleic acid, e.g., an shRNA, and expressed conditionally and/or tissue specifically using appropriate conditional/tissue specific promoters. Such an in vivo expression arrangement of the priming agent allows for the temporally priming of a particular tissue. Accordingly, only those cells in need of being targeted for RNAi/gene silencing will be primed and responsive. An RNAi/gene silencing agent is then administered, e.g., an siRNA specific for a gene target in need of knock-down is administered, e.g., intravenously or intraperitonealy. The targeted gene, e.g., a cancer gene, is then monitored using, e.g., PCR to confirm knock-down by RNAi mediated degradation.

Accordingly, in vivo priming of mammalian cells allows for the efficient and specific application of RNAi/gene silencing techniques in a whole animal.

### EXAMPLE 3

### High Throughput Screening Assays Using Activated Mammalian RISC

The following example describes methods for conducting high throughput screens for gene activities in mammalian cells using RNA interference whereby the cells (or extracts) are first primed for high levels of RISC and therefore, RNAi responsiveness.

Understanding the consequences of complex gene activities in mammalian cells is highly desirable. Previously, mammalian cells have had low responsiveness to RNAi techniques. Accordingly, mammalian cells, for example, human cells, e.g., HeLa cells are first primed using the priming agents of the invention. The primed cells (or extracts thereof) now contain high levels of RISC activity and therefore are responsive to RNAi.

To determine if the mammalian cells have been appropriately primed and are now responsive to RNAi/gene silencing techniques, the dual fluorescence efficacy assay described above can be employed. Briefly, the cells having a fluorescent GFP reporter gene, are exposed to a priming agent. The cells are then subsequently treated with an RNAi/gene silencing agent and the increase in RNAi responsiveness is determined as a function of reduced fluorescence as compared to an appropriate control.

The cells determined to be primed (or lysates thereof) are then subjected to a high throughput screen for the RNAi/gene silencing of other gene activities. Because only a handful of cells per microtitre well need be used, hundreds to thousands of different RNAi/gene silencing reactions on the primed cells can be efficiently run.

Accordingly, priming of mammalian cells allows for the efficient and specific application of RNAi/gene silencing techniques in a high throughput format.

### Equivalents

Those skilled in the art will recognize, or be able to ascertain using no more than routine experimentation, many equivalents to the specific embodiments of the invention described herein. Such equivalents are intended to be encompassed by the following claims.

1. A cell extract which mediates RNA interference (RNAi), wherein the extract is primed such that it has a high level of activated RISC relative to a suitable control.

2. The extract of claim 1, wherein the cell is of mammalian origin.

3. The extract of claim 1, wherein the cell is of human origin.

4. The extract of claim 1, wherein the cell is a HeLa cell.

5. The extract of claim 1, wherein the extract is primed with an agent selected from the group consisting of an annealed siRNA duplex, a non-annealed siRNA duplex, a single siRNA strand, and a shRNA that encodes an siRNA strand or siRNA duplex.

6. The extract of claim 1, wherein the activated RISC has about a 2-fold or greater RNA interference (RNAi) activity than RISC from a suitable control.

7. The extract of claim 1, wherein the activated RISC has about a 10-fold or greater RNA interference (RNAi) activity than RISC from a suitable control.

8. The extract of claim 1, wherein the activated RISC has about a 20-fold or greater RNA interference (RNAi) activity than RISC from a suitable control.

9. The extract of claim 1, wherein the extract is a mammalian cell extract.

10. The extract of claim 1, wherein the extract is a HeLa cell extract.

11. An extract comprising a high level of activated RISC derived from a mammalian cell having been exposed to a sufficient amount of a priming agent to achieve activated RISC.

12. The extract of claim 11, wherein the extract is a HeLa cell extract.

13. A priming agent suitable for activating RISC in a cell selected from the group consisting of an annealed siRNA duplex, a non-annealed siRNA duplex, a single siRNA strand, and a shRNA that encodes an siRNA strand or siRNA duplex.

14. A composition comprising the priming agent of claim 13, and a pharmaceutically acceptable carrier.

15. A liposome comprising the priming agent of claim 13.

16. A vector encoding the priming agent of claim 13.

17. The vector of claim 16, further comprising at least one element which mediates conditional expression.

18. The vector of claim 17, comprising a tet operator and operon.

19. A cell comprising the vector of claim 16, 17, or 18.

20. The cell of claim 19, wherein the vector is chromosomally integrated.

21. An organism comprising the cell of claim 19 or 20.

22. A kit for mediating RNA interference (RNAi) comprising,
at least one component selected from the group consisting of a priming agent, an extract having activated RISC, a cell having activated RISC, and an organism having activated RISC, and
instructions for use.

23. The kit of claim 22, wherein the priming agent is selected from the group consisting of an annealed siRNA duplex, a non-annealed siRNA duplex, a single siRNA strand, and a shRNA that encodes an siRNA strand or siRNA duplex.

24. The kit of claim 23, wherein the extract is a mammalian cell extract.

25. The kit of claim 23, wherein the cell is of mammalian origin.

26. A cell having activated RISC produced by a process comprising, exposing the cell to a sufficient amount of priming agent to activate the RISC, such that a high level of activated RISC, relative to a suitable control, is achieved.

27. The cell of claim 26, wherein the priming agent is selected from the group consisting of an annealed siRNA duplex, a non-annealed siRNA duplex, a single siRNA strand, and a shRNA that encodes an siRNA strand or siRNA duplex.

28. A cell comprising a priming agent capable of activating RISC to a high level as compared to a suitable control.

29. A cell having activated RISC, the cell having been exposed to a sufficient amount of priming agent to achieve activated RISC.

30. The cell of claim 26, wherein the cell is of mammalian origin.

31. The cell of claim 26, wherein the cell is of human origin.

32. The cell of claim 26, wherein the cell is of HeLa cell origin.

33. An extract derived from the cell of claim 26.

34. An organism comprising the cell of claim 30 or 31.

35. A method of making a cell having activated RISC, the method comprising, exposing the cell to a sufficient amount of priming agent to activate the RISC, such that a high level of activated RISC, relative to a suitable control, is achieved.

36. A method of making a cell extract having activated RISC, the method comprising, exposing a cell to a sufficient amount of priming agent to activate RISC, and extracting lysates from the activated cell.

37. A method of making activated RISC, the method comprising,
exposing a cell or cell extract to a sufficient amount of priming agent to activate RISC, and
optionally, purifying or partially purifying the activated RISC or components thereof.

38. A method of mediating RNAi, the method comprising,
contacting RISC, an extract, a cell, or an organism to a priming agent, and
exposing the RISC, an extract, cell, or organism to an siRNA such that a target specific RNAi is capable of being achieved.

39. The method of claim 38, wherein the extract is derived from a cell of mammalian origin.

40. The method of claim 38, wherein the extract is derived from a cell of human origin.

41. The method of claim 38, wherein cell is of mammalian origin.

42. The method of claim 38, wherein cell is of human origin.

43. The method of claim 38, wherein the organism is selected from the group consisting of C. elegans, Drosophila, mouse, and human.

44. A method of treating a disease or disorder associated with the activity of a protein specified by a target mRNA in a subject comprising,
administering to the subject a priming agent sufficient to activate RISC in one or more cells and
administering an siRNA in an amount sufficient for degradation of the target mRNA to occur, thereby treating the disease or disorder associated with the polypeptide encoded by the target mRNA.

45. A method of deriving information about the function of a gene in a extract, cell, or organism comprising,
exposing a primed extract, cell, or organism of any one of the preceding claims to an siRNA, maintaining the lysate, cell, or organism under conditions such that target-specific RNAi can occur,
determining a characteristic or property of the extract, cell, or organism, and
comparing the characteristic or property to a suitable control, the comparison yielding information about the function of the gene.

46. The method of claim 45, wherein the organism is selected from the group consisting of C elegans, Drosophila, mouse, and human.
